(19) United States
(12) Patent Application Publication
(10) Pub. No.: US 2025/0331832 A1
(43) Pub. Date: Oct. 30, 2025

(54) MEDICAL DELIVERY DEVICES, ASSEMBLIES, AND RELATED METHODS

(71) Applicant: Boston Scientific Medical Device Limited, Galway (IE)

(72) Inventor: Deepak Kumar SHARMA, Muzaffarnagar (IN)

(73) Assignee: Boston Scientific Medical Device Limited, Galway (IE)

(21) Appl. No.: 19/187,188

(22) Filed: Apr. 23, 2025

Related U.S. Application Data
(60) Provisional application No. 63/638,682, filed on Apr. 25, 2024.

(51) Int. Cl. A61B 17/00

(52) U.S. Cl. CPC A61B 17/00491; A61B 2017/00367; A61B 2017/00862; A61B 2017/00867

(57) ABSTRACT

A medical device may include a sheath, an actuation wire extending through the sheath and movable relative to the sheath, and a grasping portion affixed to a distal end of the actuation wire. The grasping portion may include a plurality of members configured to receive a capsule and proximal movement of the actuation wire may be configured to move at least a proximal portion of the grasping portion within the sheath in order to deploy a fluid from the capsule.

### CROSS-REFERENCE TO RELATED APPLICATION(S)

This application claims the benefit of priority under 35 U.S.C. § 119 from U.S. Provisional Application No. 63/638,682, filed on Apr. 25, 2024, which is incorporated by reference herein in its entirety.

### TECHNICAL FIELD

The disclosure relates generally to systems, devices, and methods for delivering one or more fluids. More specifically, aspects of the disclosure pertain to systems, devices, and/or methods for delivering viscous fluid that may include one or more treatment agents to a target site, via medical devices, such as endoscopes.

### BACKGROUND

Bleeding ulcers or other wound sites may occur, for example, in a subject's gastrointestinal (GI) tract. For example, following another diagnostic or treatment procedure, such as endoscopic mucosal resection (EMR) or endoscopic submucosal dissection (ESD), bleeding may be desired to be prevented or treated. In another example, a line of sutures in a GI tract may be in need of reinforcement. In a further example, a fistula may require treatment. In yet another example, peroral endoscopic myotomy (POEM) sites may need closure. Therefore, a need exists for systems, devices, and methods for delivering one or more fluids.

### SUMMARY

The disclosure includes systems, devices, and methods for delivering one or more fluids such as treatment agents to a target site of a subject, for example, to help heal an ulcer and/or to perform hemostasis. Each of the aspects disclosed herein may include one or more of the features described in connection with any of the other disclosed aspects.

For example, a medical device may include a sheath, an actuation wire extending through the sheath and movable relative to the sheath, and a grasping portion affixed to a distal end of the actuation wire. The grasping portion may include a plurality of members configured to receive a capsule and proximal movement of the actuation wire may be configured to move at least a proximal portion of the grasping portion within the sheath in order to deploy a fluid from the capsule.

Any of the systems, devices, and methods disclosed herein may include any of the following features. Each of the plurality of members may include a proximal portion and a distal portion and the distal portions of the plurality of members may extend approximately parallel to one another. The proximal portion of each of the plurality of members may extend radially outward in a distal direction. Each member of the plurality of members may include a wire. In a configuration in which the capsule is received by the plurality of members, distalmost ends of the plurality of members may be proximal of a distalmost end of the capsule. The plurality of members may have a shape-memory property. The fluid may be a first fluid and the capsule may be configured to further contain a second fluid. The medical device may further include a handle having an actuator configured to move the actuation wire proximally. In a first configuration, a distalmost end of the actuation wire may be distal to a distalmost end of the sheath. In the first configuration, proximalmost ends of the plurality of members may be distal to the distalmost end of the sheath. In a second configuration, a distalmost end of the actuation wire and proximalmost ends of the plurality of members may be proximal to the distalmost end of the sheath. The sheath may include a coil. The plurality of members may be woven. The grasping portion may further include a sleeve and the plurality of members may be coupled to the sleeve. Proximal ends of the plurality of members may be coupled to the sleeve.

This disclosure further includes a medical system including a medical device, the medical device including a sheath, an actuation wire extending through the sheath and movable relative to the sheath, and a grasping portion affixed to a distal end of the actuation wire. The medical system may further include a capsule removably received by the grasping portion. Proximal movement of the actuation wire may be configured to move at least a proximal portion of the grasping portion and a proximal portion of the capsule within the sheath in order to deploy a fluid from the capsule.

Any of the systems, devices, and methods disclosed herein may include any of the following features. The grasping portion may include a plurality of members surrounding the capsule. The capsule may include a cylindrical central portion and two tapered end portions.

This disclosure further includes a medical device, including a handle, including an actuator, a shaft extending from the handle, the shaft including (i) a coil and (ii) a wire disposed within the coil. The wire may translate along a longitudinal axis relative to the coil based on actuation of the actuator. The medical device may further include a grasping portion affixed to a distal end of the wire and configured to removably receive a capsule. Actuation of the actuator may transition the grasping portion from a first configuration to a second configuration and in the first configuration, a distalmost end of the wire and a proximalmost end of the grasping portion may be distal to a distalmost end of the coil and in the second configuration, the distalmost end of the wire and the proximalmost end of the grasping portion may be proximal to the proximalmost end of the coil.

Any of the systems, devices, and methods disclosed herein may include any of the following features. In the second configuration, a volume of the capsule may be smaller than in the first configuration.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts a perspective view of an exemplary medical device.

FIG. 2A depicts a fluid delivery capsule of the medical device.

FIG. 2B depicts an alternative fluid delivery capsule of the medical device.

FIG. 3A depicts a distal portion of the medical device in a first configuration.

FIG. 3B depicts a distal portion of the medical device in a second configuration.

FIG. 4 depicts a distal portion of an alternative medical device.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

A fluid may be used to prevent or treat bleeding or other conditions in a GI tract. In examples, a highly viscous fluid may be delivered to the wound site(s) to form a protective layer that helps to treat or minimize delayed bleeds, potential perforations, and stricture formations. Conventional devices for fluid delivery may include one or more fluid channels, with the one or more fluid channels each having a small diameter. Highly viscous fluids may require a large amount of force or pressure to flow through narrow fluid channels. Common fluid delivery devices, e.g., syringes, generally deliver fluid when a push force, e.g., a distal force, is applied to an actuator feature of such devices. Applying a large amount of force or pressure to urge the viscous fluid distally may be difficult and/or time-consuming for the user. Additionally, applying the large amount of force or pressure may increase the risk of one or more components of the medical device deforming, breaking, or otherwise failing.

Aspects of this disclosure seek to improve and ease a user's ability to deliver a highly viscous fluid via a device delivered through a working channel of an insertion device, such as a medical scope (e.g., endoscope). Additionally, various aspects of this disclosure may help the user perform wound treatment and/or hemostasis within the subject, reduce overall procedure time, reduce overall procedure costs, etc. Each of the embodiments of this disclosure is configured to deliver viscous fluid when a pull force, i.e., a proximal force, is applied to a respective feature. The presence of a pull feature for fluid delivery, as opposed to a push feature for fluid delivery, may allow for an easier application of force and/or a sufficient amount of force to deliver viscous fluid through the various openings and channels of the embodiments of this disclosure. Furthermore, such fluids may be disposed at a distal end of the device and may not be required to travel through a full length of the medical device (e.g., through a working channel of an insertion device). While the disclosure primarily relates to highly viscous fluids on the order of 200 centipoise or greater, one of ordinary skill in the art that the disclosure may be applicable to fluids of any viscosity.

Examples of fluids (e.g., biocompatible viscous fluids) that may be delivered using the devices disclosed herein include, but are not limited to, fibrin, thrombin, fluids including calcium salts, cyanoacrylates, albumin and glutaraldehyde, poly(ethylene glycol) (PEG), polyurethane, etc. Such fluids may be endoscopically delivered adhesives or other agents that help to create a protective layer that minimizes delayed bleeds, potential perforations, and stricture formations.

One of ordinary skill in the art will appreciate that the systems, devices, and methods of this disclosure may be used with a variety of biocompatible fluids and that the systems, devices, and methods of the disclosure may be applicable to various medical procedures beyond bleeding control.

Various aspects of this disclosure relate to delivery and/or mixing of various hemostatic agents at a treatment site. For example, a user may deploy multiple different hemostatic agents at the target treatment site via the systems, devices, and methods disclosed herein. In an example, a first agent (Part A) and a second agent (Part B) may be mixed together in a predefined ratio at the target treatment site. Part A and Part B may crosslink when they come in contact with one another. The cross-linked structure of the resulting combination agent (A+B) may have enhanced hemostatic properties compared to agents A or B individually. In a non-limiting example, Part A may be fibrinogen (e.g., lyophilised pooled human concentrate) and Part B may be thrombin (e.g., of bovine or human origin). Fibrinogen and thrombin may be mixed immediately before application at a target treatment site. The biocompatible fibrinogen and thrombin mixture may also contain calcium salts. One of ordinary skill in the art will appreciate that such a hemostatic mixture may be used at various locations in the human body, such as the stomach, esophagus, and colon.

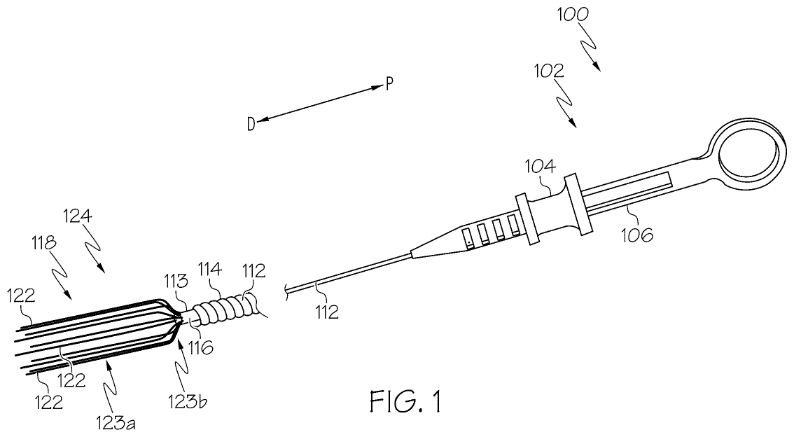

FIG. 1 depicts a perspective view of an exemplary medical device 100. It will be understood that the figures shown in the disclosure may not be to scale. For example, a distal portion of medical device 100 may be enlarged to exaggerate and emphasize certain features. As shown in FIG. 1, medical device 100 may feature a shaft 112. Shaft 112 may define a longitudinal axis and may extend from a handle 102. Shaft 112 may translate proximally and/or distally through tortuous patient anatomy via an articulable scope device such as an endoscope (not shown).

Medical device 100 may feature a grasping portion 118 located at a distal end of shaft 112. Grasping portion 118 may be similar to a basket. Grasping portion 118 may include a plurality of expandable (e.g., flexible) members 122 configured to grasp a fluid delivery capsule. G rasping portion 118 is shown in FIG. 1 with ten total members 122, though this is only exemplary. Members 122 may vary in quantity, cross-sectional shape, length as measured from the distal end of shaft 112, radius of curvature at a proximal portion, angular separation between each of the expandable members, etc.

Some or all of grasping portion 118, including members 122 may, be include wires, cables, or lines. For example, each of members 122 may include a wire that has a distal portion 123A and a proximal portion 123B. Distal portions 123B of each of members 122 may extend approximately parallel to one another and approximately parallel to a central longitudinal axis of shaft 112 and grasping portion 118. Proximal portions 123B may extend radially outward, away from the central longitudinal axis of shaft 112/grasping portion 118, moving in a distal direction. A distance between distalmost ends of members 122 may be greater than a distance between proximalmost ends of members 122, such that grasping portion 118 has a greater distal diameter/width than proximal diameter/width. Each of members 122 may have a same shape and be positioned such that they are symmetrical about the central longitudinal axis, as shown in FIG. 1.

Grasping portion 118, including members 122 may be composed of a material with shape-memory properties, such as nitinol or other similar material(s). Grasping portion 118 may be biased in an open configuration (shown in FIG. 1), such that grasping portion 118 may accept (receive) and hold a fluid delivery capsule, described in further detail below. Grasping portion 118 may have geometry suitably configured to receive a fluid delivery capsules to deliver fluids to a target treatment site. For example, as described below, in the open configuration, grasping portion 118 may have a shape that is complementary to a shape of a fluid delivery capsule.

The shape described above is merely exemplary. In alternative grasping portion 118 may be single sheath (e.g., a sleeve-like structure). In further alternatives, grasping portions 118 may include other frames, scaffolds, meshes, or other structures.

An outer portion of shaft 112 may include a coil 114 or other type of sheath. Coil 114 may have a helical shape. Coil 114 may be a wire or rod. Coil 114 may be elastically or otherwise deformable. In some examples, shaft 112 may be biased towards a particular configuration of medical device 100. For example, coil 114 may bias shaft 112 towards the longitudinal axis.

An actuation wire 116 may extend through coil 114 of shaft 112 and may be movable relative to shaft 112. Actuation wire 116 may translate proximally and/or distally in relation to coil 114. Actuation wire 116 may be affixed to grasping portion 118 on its distal end at anchor point 113. Actuation wire 116 may be welded or otherwise immovably affixed (fixedly coupled) to grasping portion 118 at anchor point 113 (e.g., a distalmost end of actuation wire 116). For example, members 122 may be affixed to anchor point 113 of actuation wire 116. For example, proximal ends of members 122 may be welded to anchor point 113 of actuation wire 116, though this is only exemplary. Members 122 may move along the longitudinal axis as actuation wire 116 moves.

Actuation wire 116 may be affixed to an actuator 104 of handle 102 at a proximal portion of medical device 100. Handle 102 may include a handle body 106. Handle 102 may have any of the features of any medical device handle known in the art. For example, actuator 104 shown in FIG. 1 may be a spool that moves longitudinally along handle body 106. Movement of actuator 104 may cause actuation wire 116 to move relative to coil 114 (e.g., actuation wire 116 may move proximally and/or distally based on movement of actuator 104). Handle 102 may include additional or alternative actuators. One of ordinary skill in the art will appreciate that various actuators may be implemented at handle 102. It will be appreciated that while the exemplary embodiments are described with respect to a pulling force to deploy fluids, the systems, devices, and methods of the disclosure may be adapted to other actuators. Operation of medical device 100 and further features of medical device 100 are described below.

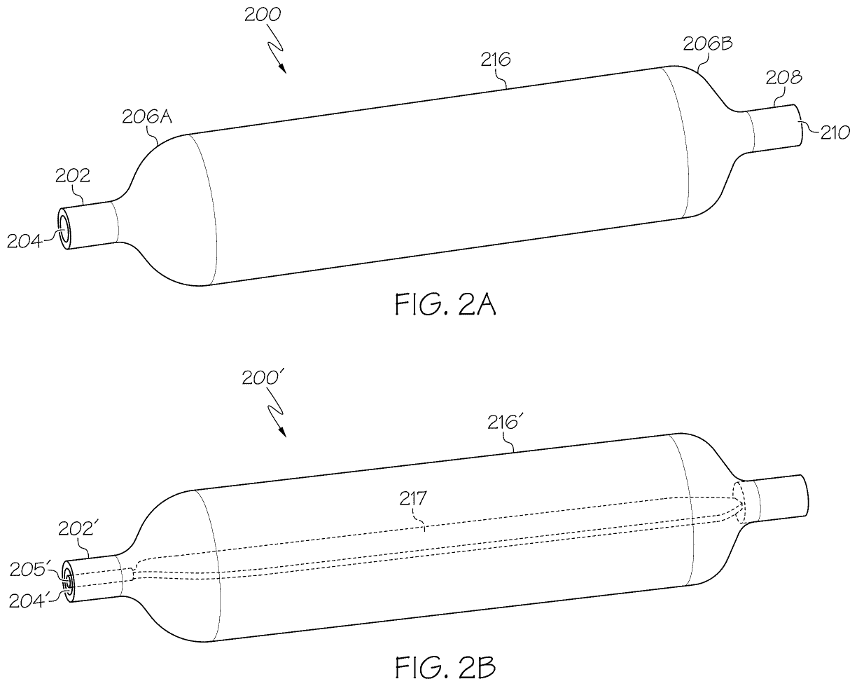

FIG. 2A shows a fluid delivery capsule 200. Capsule 200 may be inserted by a user into grasping portion 118, or capsule 200 may arrive for use pre-inserted into grasping portion 118. Capsule 200 may be removable and replaceable from grasping portion 118. One of ordinary skill in the art will appreciate that the depicted geometry of capsule 200 is only exemplary. Capsule 200 may have various external geometries, internal geometries and material compositions. For example, capsule 200 may feature an ovular cross-section, a circular-cross section, a rectangular cross section, and the like. Capsule 200 may be deformable such that force applied to capsule 200 may cause fluid to be ejected or otherwise expelled out of a hole 204 of a distal end 202 of capsule 200. In some aspects, fluid delivery capsule 200 may be elastically deformable such that fluid delivery capsule 200 returns to its original shape in the absence of an applied force via movement of shaft 112 and grasping portion 118. Capsule 200 may be removable by a user before, during, or after a medical procedure.

Capsule 200 may have an approximately cylindrical central portion 216, an approximately cylindrical distal end 202, and an approximately cylindrical proximal end 208. Distal end 202 and proximal end 206 may have a smaller cross-sectional width/diameter than central portion 216. In some examples, distal end 202 and proximal end 208 may have a same length and cross-sectional width/diameter. Alternatively distal end 202 and proximal end 206 may have different shapes.

Capsule 200 may feature curved portions 206A and 206B at distal and proximal portions of capsule 200 respectively. Curved portions 206A and 206B may extend between central portion 216 and distal end 202 and proximal end 208, respectively. Curved portions 206A and 206B may vary in cross-sectional width from an outer diameter equal to central portion 216 of capsule 200 to a smaller cross-sectional width of proximal end 208 of capsule 200 and/or distal end 202 of capsule 200. In other words, curved portion 206A may taper radially inward in a distal direction. Curved portion 206B may taper radially inward in a proximal direction. Thus, capsule 200 may include a cylindrical central portion 216 and two tapered end portions 206A, 206B. While curved portions 206A and 206B are depicted as mirror images in FIG. 2A, this is only exemplary. In some aspects, curved portion 206A may feature a different cross-sectional shape than curved portion 206B. In some aspects, curved portions 206A and 206B may not be present, such that central portion 216 forms distal and proximal ends of capsule 200. In other aspects, portions 206A and 206B may have cone- or frustum-like shapes.

It will be understood that any portion of fluid delivery capsule 200 may be shaped to correspond to grasping portion 118. For example, as discussed in further detail below, proximal end 208 may be shaped to conform to the geometry of grasping portion 118. Proximal end 208 may terminate proximally with a surface 210 that is approximately perpendicular to the longitudinal axis.

Hole 204 may have various geometries. For example, hole 204 may feature a circular cross section. In some aspects, hole 204 may be a plurality of holes such that fluid evacuates capsule 200 through the plurality of holes. One of ordinary skill in the art will appreciate that various geometries of hole 204 may be implemented with capsule 200.

FIG. 2B depicts an alternative fluid delivery capsule 200′. Capsule 200′ may be substantially similar to capsule 200 and may feature any feature described with respect to capsule 200 except as escribed below. In examples, fluid delivery capsule 200′ may have internal features in central portion 216′ (or other portions) configured to separate fluids prior to deployment. Fluid delivery capsule 200′ may have an internal partition 217 separating Part A and Part B of a treatment agent, discussed above. Internal partition 217 may be a flexible membrane (e.g., rubber or a similar elastomer) configured to bend with deformation of capsule 200′. The internal partition may terminate at a distal end 202′ of the fluid delivery capsule 200′ such that squeezing on the fluid delivery capsule 200′ may cause fluid to eject or otherwise evacuate out of fluid delivery capsule 200′ along separate channels defined by a distal divider 205′, and out of hole 204′ due to deformation of fluid delivery capsule 200′ reducing the internal volume of capsule 200′ for fluid(s) to occupy (e.g., is less volume than the first configuration). In some aspects, distal divider 205′ may be coupled to (e.g., formed integrally with) internal partition 217. In some aspects, distal divider 205′ may be helically shaped to improve fluid mixing outside of capsule 200′.

In yet another example, fluid delivery capsules 200 or 200′ may feature one or more sub-capsules. The one or more sub-capsules may each contain one or more fluids. For example, a first sub-capsule may contain Part A, and a second sub-capsule may contain Part B. When force is applied to fluid delivery capsule 200 or 200′, the sub-capsules may release fluid to be ejected out of distal end 202 or 202′ via hole 204 or 204′.

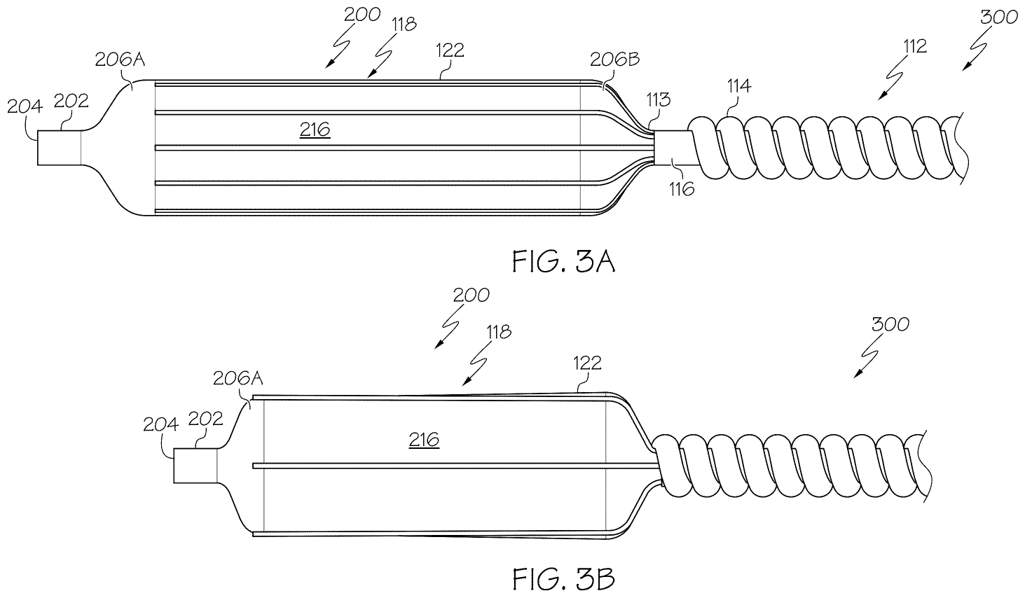

FIG. 3A depicts a distal portion of the medical device in a first configuration. For ease of reference, capsule 200 grasped by grasping portion 118 may also be referred to assembly 300. The first configuration may be a “default” configuration. The first configuration may correspond to assembly 300 before a user deploys fluid at a target site. The first configuration may correspond to the assembly 300 during movement through tortuous patient anatomy. FIG. 3A may correspond to the state of assembly 300 at the target treatment site before deployment of one or more fluids. For example, a user may position distal end 202 at a suitable distance from a bleed at the target treatment site while the assembly 300 is in the first configuration. In FIG. 3A, a portion of actuation wire 116 is exposed (e.g., is not surrounded by coil 114) distal to a distalmost end of coil 114.

FIG. 3A shows capsule 200 surrounded by (received by) and grasped by grasping portion 118 (e.g., via members 122). Members 122 may provide a grasping, inward force to capsule 200 (e.g., approximately transverse to the longitudinal axis) such that capsule 200 stays in place during deployment to the target site, but not enough grasping force such that fluid is ejected out of hole 204 in the absence of proximal force applied to actuation wire 116, as described above. In a non-limiting example, ten members 122 may be approximately spaced 36° apart from one another (e.g., 360°÷10), relative to the outer diameter of the circular cross section of central portion 216.

As shown in FIG. 3A, proximal portions 123B of members 122 may receive and extend around proximal curved portion 206B. A shape of proximal portions 123B of members 122 may be complementary to a shape of proximal curved portion 206B. In other words, a proximal portions 123B and curved portion 206B may taper radially outward in a distal direction at approximately the same angle. Distal portions 123A of members 122 may extend along central portion 216. Distalmost ends of distal portions 123A may terminate along central portion 216, proximal of distal curved portion 206A and proximal of a distalmost end of capsule 200. In alternatives, members 122 may have portions that are distal to distal portions 123A and taper radially inwardly along distal curved portion 206A. In some aspects, a distal portion of actuation wire 116 may be received (e.g., mate with) by a cavity at a proximalmost end of proximal end 208 of capsule 200. In some aspects, a distal end of actuation wire 116 may include a cavity for receiving proximal end 208 of capsule 200.

FIG. 3B depicts a distal portion of the medical device in a second configuration. FIG. 3B may depict the “deployed” or partially deployed (e.g., second) state of assembly 300. To transition assembly 300 from the first state to the second state the user may pull actuation wire 116 proximally via actuator 104. For example, the user may slide actuator 104 proximally, which may pull actuation wire 116 proximally. As discussed above, actuation wire 116 may be affixed to members 122 at anchor point 113. Proximal movement of actuation wire 116 may thus proximally move grasping portion 118 and capsule 200 relative to coil 114, due to the grasping force applied to capsule 200 via members 122.

As capsule 200 moves proximally, it may contact coil 114. A portion of actuation wire 116 depicted in FIG. 3A as being distal of coil 114 may be concealed by coil 114 in the second configuration. In other words, a distalmost end of actuation wire 116 may be proximal of a distalmost end of coil 114. Proximalmost portions of proximal portions 123B of members 122 may also be received within coil 114 in the second configuration. Thus, at least a portion of grasping portion 118 may be within coil 114. Increasingly distal portions of members 122 may be squeezed (e.g., enter) into coil 114 as members 122 are pulled proximally by actuation wire 116. Deformation of portions of members 122 as they are moved within coil 114 may squeeze (e.g., apply a force transverse to the longitudinal axis) capsule 200. Proximal portions of capsule 200 (e.g., a proximal end of curved portion 206B) may be drawn within coil 114 such that fluid(s) A contained within capsule 200 may be forced out of distal hole 204.

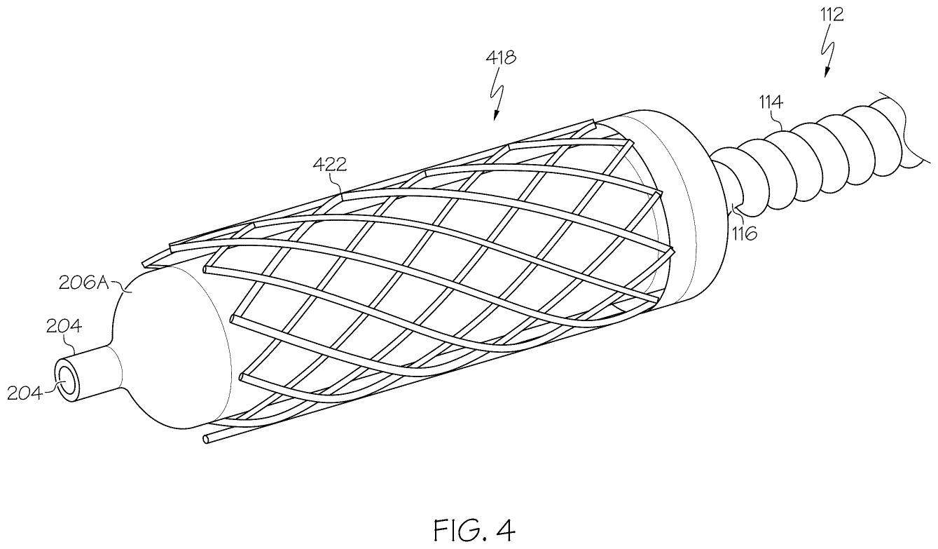

As shown in FIG. 4, capsule 200 may expand radially or otherwise deform in response to proximal portions of capsule 200 being drawn into coil 114. As a result, members 122 may shift their position relative to one another in response to the force applied by coil 114 on capsule 200. For example, members 122 may bulge radially outward and/or have a greater distance between adjacent members 122. Deformation of capsule 200 may reduce the available internal volume of capsule 200 for fluids, which may cause fluid A to be ejected through hole 204 at the target site.

FIG. 4 shows a distal portion of a medical device 400 having an alternative grasping portion 418, according to aspects of the disclosure. Device 400 may have any of the features of device 100, except grasping portion 418 may be used in place of grasping portion 118. Grasping portion 418 may feature a plurality of woven, knit, and/or latticed members 422. For example, grasping portion 418 may be similar to a stent. Latticed members 422 may have similar functionality to members 122 discussed above. Latticed members 422 may be wires with circular cross sections, though this is only exemplary. In some examples, latticed members 422 may have a coating or covering similar to a coated stent.

Grasping portion 418 may also include a proximal sleeve 450 that is coupled to actuation wire 116 (not shown in FIG. 4 but depicted in previous Figures). Sleeve 450 may be flexible and may have a complementary shape to a proximal portion of capsule 200. For example, proximal sleeve 450 may have a funnel-like shape. Alternatively, sleeve 450 may be omitted and members 422 may be directly coupled to actuation wire 116.

A user may insert capsule 200 into grasping portion 418 at a distal end of grasping portion 418. Members 422 may extend along central portion 216 of capsule 200. Proximal sleeve 450 may extend along curved portion 206B (not shown in FIG. 4). Distal curved portion 206A may extend distally of grasping portion 418. Sleeve 450 and/or latticed members 422 may apply squeezing force to capsule 200 to deploy fluid from capsule 200 when actuation wire 116 is moved proximally, as discussed above for device 100. As compared with members 122, members 422 may exert a more uniform force. One of ordinary skill in the art will appreciate that grasping portion 418 is only exemplary and that various possible configurations of latticed members 422 may be possible.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical device comprising:
a sheath;
an actuation wire extending through the sheath and movable relative to the sheath; and
a grasping portion affixed to a distal end of the actuation wire, wherein the grasping portion includes a plurality of members configured to receive a capsule, and wherein proximal movement of the actuation wire is configured to move at least a proximal portion of the grasping portion within the sheath in order to deploy a fluid from the capsule.

2. The medical device of claim 1, wherein each of the plurality of members includes a proximal portion and a distal portion, wherein the distal portions of the plurality of members extend approximately parallel to one another.

3. The medical device of claim 2, wherein the proximal portion of each of the plurality of members extends radially outward in a distal direction.

4. The medical device of claim 1, wherein each member of the plurality of members includes a wire.

5. The medical device of claim 1, wherein, in a configuration in which the capsule is received by the plurality of members, distalmost ends of the plurality of members are proximal of a distalmost end of the capsule.

6. The medical device of claim 1, wherein the plurality of members have a shape-memory property.

7. The medical device of claim 1, wherein the fluid is a first fluid, wherein the capsule is configured to further contain a second fluid.

8. The medical device of claim 1, further comprising a handle having an actuator configured to move the actuation wire proximally.

9. The medical device of claim 1, wherein, in a first configuration, a distalmost end of the actuation wire is distal to a distalmost end of the sheath.

10. The medical device of claim 9, wherein, in the first configuration, proximalmost ends of the plurality of members are distal to the distalmost end of the sheath.

11. The medical device of claim 10, wherein, in a second configuration, a distalmost end of the actuation wire and proximalmost ends of the plurality of members are proximal to the distalmost end of the sheath.

12. The medical device of claim 1, wherein the sheath includes a coil.

13. The medical device of claim 6, wherein the plurality of members are woven.

14. The medical device of claim 13, wherein the grasping portion further includes a sleeve, and wherein the plurality of members are coupled to the sleeve.

15. The medical device of claim 14, wherein proximal ends of the plurality of members are coupled to the sleeve.

16. A medical system comprising:
a medical device, including:
a sheath;
an actuation wire extending through the sheath and movable relative to the sheath; and
a grasping portion affixed to a distal end of the actuation wire; and
a capsule removably received by the grasping portion, wherein proximal movement of the actuation wire is configured to move at least a proximal portion of the grasping portion and a proximal portion of the capsule within the sheath in order to deploy a fluid from the capsule.

17. The medical system of claim 16, wherein the grasping portion comprises a plurality of members surrounding the capsule.

18. The medical system of claim 16, wherein the capsule includes a cylindrical central portion and two tapered end portions.

19. A medical device, comprising:
a handle, including an actuator;
a shaft extending from the handle, the shaft comprising (i) a coil and (ii) a wire disposed within the coil, wherein the wire may translate along a longitudinal axis relative to the coil based on actuation of the actuator; and
a grasping portion affixed to a distal end of the wire and configured to removably receive a capsule, wherein actuation of the actuator transitions the grasping portion from a first configuration to a second configuration, wherein, in the first configuration, a distalmost end of the wire and a proximalmost end of the grasping portion are distal to a distalmost end of the coil and wherein, in the second configuration, the distalmost end of the wire and the proximalmost end of the grasping portion are proximal to the proximalmost end of the coil.

20. The medical device of claim 19, wherein, in the second configuration, a volume of the capsule is smaller than in the first configuration.
